(19) United States
(12) Patent Application Publication
(10) Pub. No.: US 2017/0231603 A1
(43) Pub. Date: Aug. 17, 2017

(54) Oral Sampling Swab And Uses Thereof

(71) Applicant: The Procter & Gamble Company, Cincinnati, OH (US)

(72) Inventors: Jinman Liu, Beijing (CN); Ricardo Leal-Macias, Beijing (CN)

(21) Appl. No.: 15/405,342

(22) Filed: Jan. 13, 2017

(30) Foreign Application Priority Data
Jan. 13, 2016 (WO) CN2016/070761

(51) Int. Cl. A61B 10/00; A61B 5/00; C09B 19/00

(52) U.S. Cl. CPC A61B 10/0051; C09B 19/00; A61B 5/4547; A61B 2010/0006

(57) ABSTRACT

Oral sampling swab for visually assessing treatment effectiveness of an oral care product in decreasing the amount of bacteria in an oral cavity and method of use thereof.

### FIELD OF THE INVENTION

The invention is directed to an oral sampling swab and methods for using the swab to allow consumers to visually assess treatment effectiveness of an oral care product.

### BACKGROUND OF THE INVENTION

Demonstrations are effective and inexpensive means to promote and market new products or new benefits of existing products to the consumers. In particular, demonstrations can be used to determine degree of oral cleanliness of an oral cavity, for example, by measuring the amount of bacteria present in a biological sample (e.g., saliva, plaque) collected from the oral cavity of a subject. When the amount of bacteria is high in the obtained biological sample, the oral cleanliness is considered to be poor, and the cleaning efficacy of the oral care product is considered to be low. Conversely, when the amount of bacteria is low in the obtained biological sample, the oral cleanliness is considered to be good, and the cleaning efficacy of the oral care product is considered to be high.

Regardless of whether the demonstration is live (i.e., with a presenter) or standalone (i.e., self-evaluation), it is desirable that the demo articles used in the demonstrations be portable to various sites, easy to use with minimal instructions required, easy to understand the results with minimal explanation, and/or take up minimal display space. Unfortunately, these properties can be difficult to achieve when the demonstrations involve the oral cavity.

Sampling swabs are well-known tools for collecting samples to assess for the presence and/or levels of contaminants (e.g., bacteria) deposited on surfaces of an individual, package or bag. For example, CN Patent No. CN102634448B (Ningbo) and CN Utility Model No. CN202530081U (Ningbo) disclose sampling swabs for collecting samples from conventional surfaces (e.g., hard surfaces) and transferring the samples into a test liquid containing luciferase fluorescence to detect the number of bacteria within the test liquid by an ultraviolet color change. For another example, CN Utility Model No. CN201949050U (Ningbo) discloses a sampling swab having a swab head made of plastic nylon for improved collection of epithelial cells from an oral cavity and transferring the obtained sample to a laboratory for measurement. Unfortunately, those sampling swabs are not designed to obtain biological samples from an oral cavity or if they are intended for the oral cavity are not suitable to collect biological samples (e.g., saliva, plaques) related to oral cleanliness. Furthermore, those sampling swabs tend to require heavy and sensitive detection equipment or the like to measure the presence and/or level of bacteria or other biological materials. The equipment are difficult to transport and set-up, which make them not ideal for use in demonstrations. Furthermore, those sampling swabs may require advanced examination techniques by a technical expert, doctor or the like, thus leaving room for improvement.

Accordingly, the need exists for an improved method and oral sampling swab for consumers to visually assess the treatment effectiveness (i.e., oral cleanliness) of an oral care product in an oral cavity. It is advantageous that the method and oral sampling swab is easy to use and convenient for the consumers to interpret the results without significant technical assistant. It is advantageous that the method and oral sampling swab are easily transportable to various sites to be used in demonstrations to consumers, without the need for additional heavy and sensitive detection equipment. It is also advantageous that the method and oral sampling swab are predictive of clinical effectiveness for treatment with the oral care product. It is further advantageous that the method and oral sampling swab of the present invention can be used as a quick screening tool for oral care actives.

### SUMMARY OF THE INVENTION

In a first aspect, the present invention is directed to an oral sampling swab for assessing treatment effectiveness of an oral care product in decreasing the amount of bacteria in an oral cavity, the oral sampling swab comprising:

(a) a swab comprising a handle at one end for handling the swab, a swab head, and a hollow swab stem connecting the handle to the swab head to provide fluid passage of a dye stored in a receptacle inside the handle to the swab head, wherein the swab head is configured for obtaining a biological sample from an oral cavity, wherein the dye is configured to exhibit a visible color change upon reacting with bacteria contained in the obtained biological sample; and

(b) a reaction tube comprising an upper open end and an opposing lower closed end, wherein the swab is removably inserted in the reaction tube such that at least the handle of the swab protrudes from the upper open end when the swab head abuts the lower closed end of the reaction tube.

In another aspect, the invention provides for a method of providing to a consumer a visual demonstration of the treatment effectiveness of an oral care product in decreasing the amount of bacteria in an oral cavity, the method comprising the steps of:

(a) providing an oral sample swab, as set forth herein;

(b) removing the swab from the reaction tube;

(c) obtaining a biological sample of an oral cavity with the removed swab;

(d) returning the swab to the reaction tube with the obtained biological sample;

(e) releasing the dye from the stored receptacle of the returned swab to the reaction tube so as to immerse the swab head in the dye; and

(f) assessing whether the dye exhibits a visible color change or degree of visible color formation as indicative of bacteria amount and a positive assessment of the oral care product's treatment effectiveness.

In another aspect, the invention provides a kit for providing to a consumer an assessment of treatment effectiveness of an oral care product in decreasing the amount of bacteria in an oral cavity comprising the oral sampling swab, as set forth herein, and optionally user instructions.

These and other features of the present invention will become apparent to one skilled in the art upon review of the following detailed description when taken in conjunction with the appended claims.

### BRIEF DESCRIPTION OF THE DRAWINGS

While the specification concludes with claims particularly pointing out and distinctly claiming the invention, it is believed that the invention will be better understood from the following description of the accompanying figures in which like reference numerals identify like elements, and wherein:

FIG. 1 shows a cross-sectional view of an oral sampling swab (1) according to an embodiment of the present invention.

FIG. 2 shows an enlarged cross-sectional view of a snap-locking mechanism (30) of the swab (1) according to an embodiment of the present invention.

FIG. 3 shows a figure with instructions for how to use the oral sampling swab (1) to test for treatment effectiveness of oral care products.

FIG. 4 shows a drawing of the oral cavity with labels for different positions to swab and obtain biological samples.

### DETAILED DESCRIPTION OF THE INVENTION

It is to be understood that the scope of the claims is not limited to the specific devices, apparatuses, methods, conditions or parameters described and/or shown herein, and that the terminology used herein is for the purpose of describing particular embodiments by way of example only and is not intended to be limiting to the claimed invention. Also, as used in the specification including the appended claims, the singular forms “a”, “an”, and “the” include the plural.

As used herein, the term “about” when placed before a numerical value “X” refers to an interval extending from 10% of X, preferably 5% of X, and even more preferably to an interval extending from 2% of X.

As used herein, any of the terms “comprising”, “having”, “containing”, and “including” means that other steps, ingredients, elements, etc. which do not adversely affect the end result can be added. Each of these terms encompasses the terms “consisting of and ” consisting essentially of . Unless otherwise specifically stated, the elements and/or equipment herein are believed to be widely available from multiple suppliers and sources around the world.

As used herein, the term “consumers” is meant to include the customers who purchase the product, users of the product, or the store owners or managers who decide whether to stock their shelves with the product.

As used herein, the term “oral care product” is meant a product, which in the ordinary course of usage, is not intentionally swallowed for purposes of systemic administration of particular therapeutic agents, but is rather retained in the oral cavity for a time sufficient to contact substantially all of the dental surfaces (i.e., tooth) and/or oral tissues for treatment of oral cavity sensitivity. The oral care product may be in various forms including toothpaste, dentifrice, tooth gel, subgingival gel, mouth rinse, mousse, foam or denture product. The term “oral care product” may also include treatment regimens where these aforementioned compositions may be applied to teeth via an implement, such as a toothbrush or the like. Suitable examples of dentifrice can be found in U.S. Patent Publication No. US2011/239736 A1. The term “oral care product” may also include individual oral care actives.

### Oral Sampling Swab

It has importantly been found that the oral sampling swab (1) of the present invention is an effective communication tool that allows consumers to visually assess the treatment effectiveness (i.e., oral cleanliness) of an oral care product in an oral cavity. It works by obtaining a biological sample (e.g., saliva, plaque) from a consumer's oral cavity and providing a visible color change to confirm the presence and/or level of bacteria in the obtained biological sample to reflect the degree of oral cleanliness of the oral cavity. Preferably, biological samples are taken pre- and post- product usage to show a correlation of the presence and/or level of bacteria in the oral cavity (i.e., oral cleanliness) to the product's cleaning efficacy. The oral sampling swab (1) advantageously works without the need for heavy or sensitive detection equipment such that it can be used by the consumers, particularly for self-evaluation. The oral sampling swab (1) also provides for a quick “a-ha” moment by the consumers of a product's performance without requiring a professional background and/or a lot of explanation, yet still be clinically meaningful enough to be accepted by professionals (e.g., dentists).

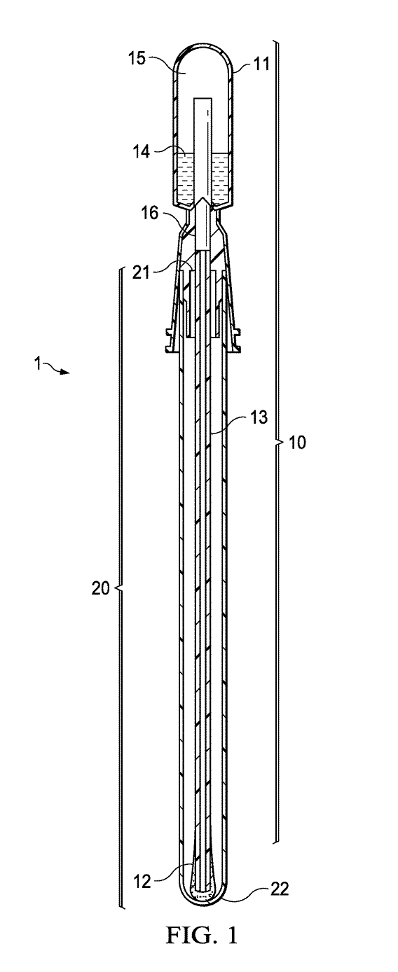

The oral sampling swab (1) can be of any dimensions so long as it is small enough to be easily transported to different sites and conveniently fits into available display space. For example, referring to the embodiment illustrated in FIG. 1, the length x diameter dimensions of the oral sampling swab (1) can be 14 cm×1.5 cm. Alternative dimensions are possible so long as the oral sampling swab (1) is not too short for convenient collection of the biological samples, preferably from the posterior region of the oral cavity. The smaller size is advantageous for transport, particular air mail, for delivery of the oral sampling swab (1) directly to consumers' home where the demonstration involves self-evaluation. The oral sampling swab (1) can be made of any materials (e.g., plastic) which can be molded or shaped, while still being durable enough to be transported without breaking. Preferably, the materials are hard plastics such as polycarbonate, polyethylene (“PE”), polypropylene (“PP”), polyvinylchloride (“PVC”) or the like. The external surface of the oral sampling swab (1) may have an optional mark, such as a product logo, linking the oral sampling swab (1) to the product to be demonstrated. Other marks, such as a pattern, a character, a logo, a word, a letter, a claim, a slogan, a picture, a photo, or a combination thereof, can be used to make the oral sampling swab (1) appear less clinical and more appealing to the consumers.

With continued reference to FIG. 1, the oral sampling swab (1) comprises a swab (10) and a reaction tube (20). The swab (10) comprises a handle (11) at one end for handling the swab (10), a swab head (12), and a hollow swab stem (13) connecting the swab handle (11) to the swab head (12). The hollow swab stem (13) has an overall length from 7 cm to 11 cm, preferably from 8 cm to 10 cm. The inventors have found that the overall length of the hollow swab stem (13) has an impact on ease with which consumers can use the oral sampling swab (1). For example, if the overall length is too long then the consumers have difficulty inserting the swab (10) into the oral cavity to obtain biological samples. However, if the overall length of the hollow swab stem (13) is too short then it will be difficult to reach the posterior regions of the oral cavity. With the recited overall length, the consumers can handle the swab (10) to easily fit the swab (10) inside the oral cavity to obtain the biological samples from all areas of the oral cavity.

The swab head (12) is designed to collect biological samples (e.g., saliva, plaque) obtained from the oral cavity. The oral cavity is selected from the gum, teeth, interface between the gum and teeth, or combinations thereof. The swab head (12) is made of a cellulosic fabric comprising of cotton, linen, rayon, or flax, or blends thereof. Preferably, the swab head (12) is cotton since it is more suitable for collecting biological samples (e.g., saliva, plaque) from the oral cavity and provides an acceptable feel in the oral cavity to the consumers. Furthermore, the cotton swab head (12) is preferred since it tends to not easily release the biological samples (e.g., saliva, plaque) once collected from the oral cavity. The swab head (12) has a diameter from 0.35 cm to 0.60 cm, preferably from 0.37 cm to 0.45 cm. The inventors have found that the diameter of the swab head (12) is important to the optimized performance of this demo tool. For example, if the diameter is too small then not enough biological samples (e.g., saliva, plaque) are collected to cause the visible color change. However, if diameter of the swab head (12) is too large then it causes discomfort to the consumers when inserted into the oral cavity. With the recited diameter, the swab head (12) can obtain sufficient amount of biological samples (e.g., saliva, plaque) to allow for the color change reaction visible to the naked eye without causing discomfort to the consumers.

The swab handle (11) further comprises a receptacle (15) inside the handle for storing a dye (14). The dye (14) is configured to exhibit a visible color change upon reacting with bacteria contained in the obtained biological samples from the oral cavity. Preferably, the dye (14) is selected from: 7-Hydroxy-3H-phenoxazin-3-one 10-oxide (“resazurin”); (3-(4,5-dimethylthiazol-2-yl)-5-(3-carboxymethoxyphenyl)-2-(4- sulfophenyl)-2H-tetrazolium) (“MTS”) (2-(4-Iodophenyl)-3-(4-nitrophenyl)-5-(2,4-disulfophenyl)-2H-tetrazolium) (“WST-1”) (2-(2-methoxy-4-nitrophenyl)-3-(4-nitrophenyl)-5-(2,4-disulfophenyl)-2H-tetrazolium) (“WST-8”) 3-[4,5 -dimethylthiazol-2-yl]-2,5-diphenyltetrazolium bromide (“MTT”); 2,3,5-triphenyltetrazolium chloride (“TTC”); 2-[-iodophenyl]-3-[4-dinitrophenyl]-5-phenyltetrazoliumchloride (“INT”); 2,3-bis[2-methoxy-4-nitro-5-sulfophenyl]-2H-tetrazolium-5-carboxanilide (“XTT”); or a salt thereof. Alternatively, the dye can be any reagent that causes a visible color change upon contact with the bacteria, with the proviso that the dye does not include a firefly luciferase.

The total volume of the dye (14) is from 275 μL to 500 μL, preferably from 300 μL to 350 μL. The concentration of the dye (14) is from 20 μg/mL to 100 μg/mL, preferably from 40 μg/mL to 60 μg/mL. Preferably, the dye (14) is resazurin. Resazurin is an oxidation-reduction indicator and is blue and weakly fluorescent by itself. Resazurin can be reduced by NADH or NADPH, generated by the metabolism of bacteria, and irreversibly converted into a visible pink color and highly red fluorescent Resorufin. This pink color change is easily visible to the naked eye, without the help of any special equipment.

In addition to the dye (14), the oral sampling swab (1) may preferably combine the dye (14) with additional reagent such as, for example, phenazine methyl sulfate (“PMS”), phenazine ethyl sulfate (“PES”), or combinations thereof. The combination of the dye (14) with the reagent, is believed to show a beneficial effect for accelerating the visible color change, particularly in reaction conditions of a short period of time and/or at room temperature, as compared with the case of not including the reagent. The concentration of the reagent is from 0.1 to 1 mM, preferably from 0.1 to 0.5 mM.

The receptacle (15) may further comprise a fragible spacer (16) configured to contain the dye (14) in the receptacle (15) before the oral sampling swab (1) is used. During use, the dye (14) is released from storage from the receptacle (15) upon sufficient pressure being applied to break the fragible spacer (16), such that the dye (14) passes from the receptacle (15) through the hollow swab stem (13) into the reaction tube (20). Preferably, the fragible spacer (16) is located at the connection between lower portion of the receptacle (15) and the upper portion of the hollow swab stem (13). Alternatively, it is possible that the fragible spacer (16) can be place in any other position along the hollow swab stem (13) so long as it is convenient for the consumer to apply pressure to break the fragible spacer (16) to release the dye (14) from storage. The advantage of having the dye (14) contained within the oral sampling swab (1) is that all of the components of the demonstrations are housed within the oral sampling swab (1). The inventors came up with the notion of an oral sampling swab (1) that comprises reagents for measuring bacteria. The oral sampling swab (1) allows the measurement to be carried out in a short period of time in one examination of the oral cavity, without the need for additional reagent and/or detection equipment.

The reaction tube (20) comprises an upper open end (21) and an opposing lower end (22). The swab (10) is removably inserted in the reaction tube (20) such that at least the handle of the swab (11) protrudes from the upper open end (21) when the swab head (12) abuts the lower closed end (22) of the reaction tube (20). The inventors have found that consumers are confused as where to check for the visible color change when the swab head (12) does not abuts the lower closed end (22) of the reaction tube (20). For example, a cause for confusion is that the visible color change in the dye (14) solution that pools at the lower closed end (22) of the reaction tube (20) may be different from the visible color change in the swab head (12). Therefore, with the arrangement of the swab head (12) abutting the closed end (22) of the reaction tube (20), the observed visible color change should be the same because the swab head (12) is immersed in the dye (14). This arrangement tends to minimize or avoid confusion to the consumers.

At least a portion of the reaction tube (20) is translucent or transparent so long as it is convenient for the consumers to view the visible color change reaction between the sampled bacteria and dye (14). Preferably, the lower closed end (22) of the reaction tube (20) is translucent or transparent since that is where the dye (14) will pool once it has been released from storage from the receptacle (15).

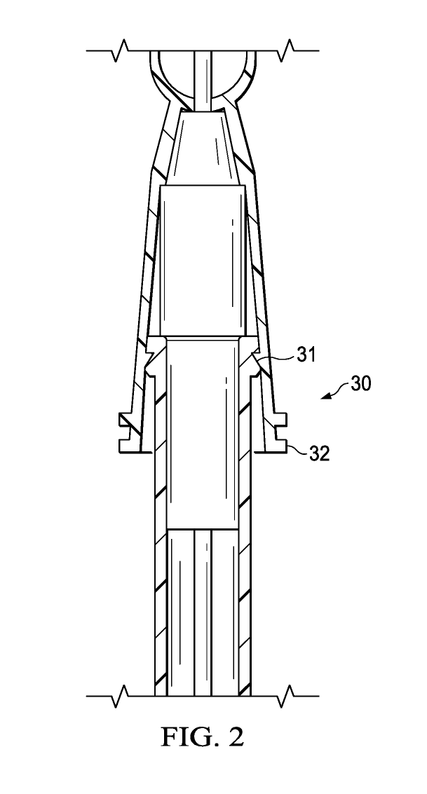

Further, the oral sampling swab (1) optionally comprises a snap-locking mechanism (30) for securing, preferably irreversibly securing, the dye (14) inside the reaction tube (20) once it has been released from the storage receptacle (15). This arrangement can be useful to comply with safety requirements for use of the dye to permit consumer self-evaluation, preferably unsupervised, with the oral sampling swab (1). With reference to FIG. 2, the snap-locking mechanism (30) is formed of a mating ring (31) in the upper end (21) of the reaction tube (20) and a lip (32) integrally formed to a lower portion of the swab handle (11), such that the insertion of the lip (32) through the locking ring (31) secures, preferably irreversibly secures, the swab (10) to the reaction tube (20). Other designs of the snap-locking mechanism (30) may be used so long as they permit securing, preferably irreversible securing, of the swab (10) to the reaction tube (20).

### Method of Use

In another aspect, the present invention is directed to a method of providing to a consumer a visual demonstration of the treatment effectiveness of an oral care product in decreasing the amount of bacteria in an oral cavity. The effectiveness of the treatment correlates to the oral cleanliness of the oral cavity.

The method according to the present invention, comprises the steps of:

(a) providing an oral sampling swab (1) as set forth above;

(b) removing the swab (10) from the reaction tube (20);

(c) obtaining a biological sample of an oral cavity with the removed swab (10);

(d) returning the swab (10) to the reaction tube (20) with the obtained biological sample;

(e) releasing the dye (14) from the stored receptacle (15) of the returned swab (10) to the reaction tube (20) so as to immerse the swab head (20) in the dye (14); and

(f) assessing whether the dye (14) exhibits a visible color change or degree of visible color formation as indicative of bacteria amount and a positive assessment of the oral care product's treatment effectiveness.

Preferably, the method, as set forth above, wherein the step (c) obtains a biological sample from at least a posterior region of the oral cavity. Preferably, the method, as set forth above, wherein the step (f) assesses the dye (14) visible color change or degree of visible color formation over a period of from 1 minute to 5 minutes, preferably from 2 minutes to 3 minutes. The oral care product is a mouth wash, strip, toothbrush, dentifrice or toothpaste, preferably a toothpaste.

The present invention also provides a kit for providing to a consumer an assessment of treatment effectiveness of an oral care product in decreasing the amount of bacteria in an oral cavity comprising the oral sampling swab (1) as set forth above. The kit may optionally include instructions for use of the oral sampling swab (1) for providing to a consumer an assessment of treatment effectiveness.

### EXAMPLE

In order that the present invention described herein may be more fully understood, the following examples are set forth. It should be understood that these examples are for illustrative purposes only and are not to be construed as limiting this invention in any manner.

### Example 1

### Visual Assessment of Treatment Effectiveness of Oral Care Product

The demonstration tool of the present invention is being utilized to show to consumers the cleaning performance of an oral care product (e.g., Crest® Pro-Health toothpaste-Complete 7 (Lot #42230386FCN, a product from the Procter & Gamble Company, Guangzhou, China)) based on the detection of bacteria in the obtained biological samples by an observed visible color change. The following protocol is used:

1. Study subjects are screened to participate in the test based on the following eligibility criteria: (i) not pregnant or nursing; and (ii) have not brushed their teeth within the last hour.

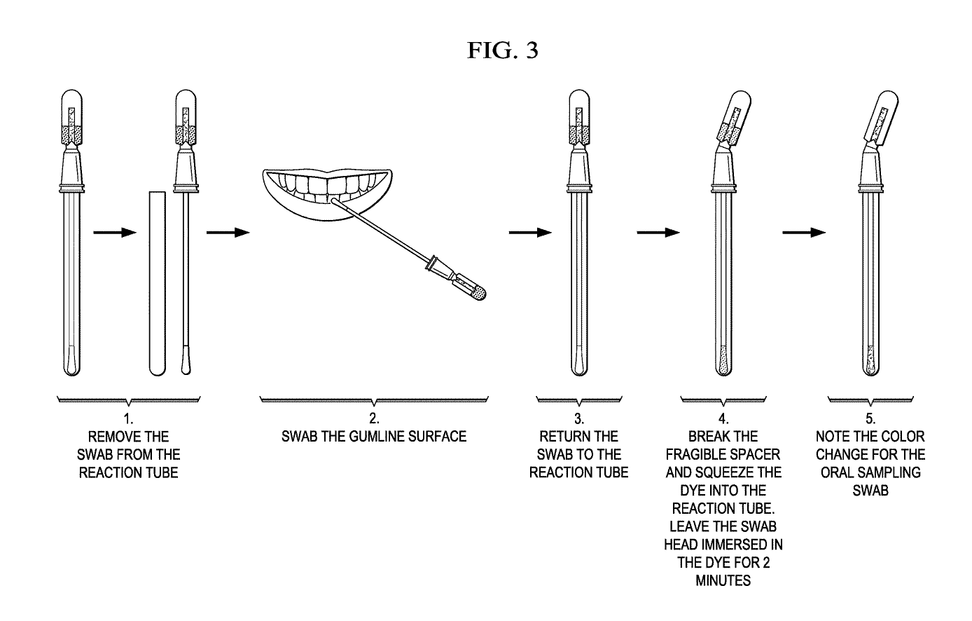

2. Give the oral sampling swab (1) and user instructions (as set forth in FIG. 3) to the subjects.

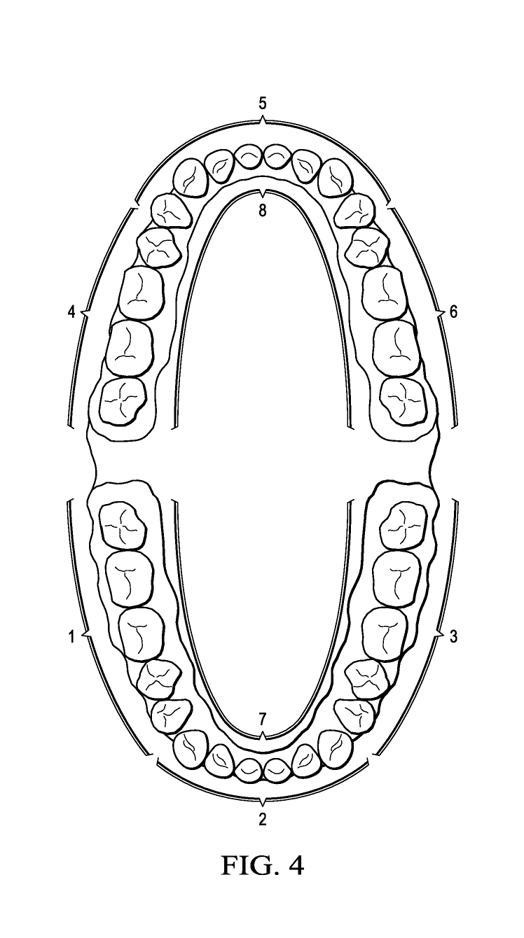

3. Request the subjects to obtain a biological sample from their oral cavity before brushing their teeth with the oral care product by following these instructions:
(a) grasp the swab handle (11) by the thumb and forefingers horizontally and remove the swab (10) from the reaction tube (20);
(b) swab the gum line surface at the 8 positions as shown in FIG. 4. For positions 1 to 6, swab back and forth twice. For positions 7 and 8, swab back and forth once; and
(c) return the swab (10) to the reaction tube (20) and label the oral sampling swab (1) as “Before Brush”.

4. Request subjects to obtain a biological sample from their oral cavity after brushing their teeth with the oral care product by following these instructions:
(a) brush teeth with Crest® Pro-Health toothpaste-Complete 7; and
(b) repeat obtaining sampling steps as in 3(a)-(c) and label the oral sampling swab (1) as “After Brush”.

5. For both oral sampling swabs (1), break the fragible spacers (16) and squeeze the dye (14) from the receptacles (15) through the hollow swab stems (13) to the reaction tubes (20).

6. Leave the swab heads (12) immersed in the dye (14) for 2 minutes.

7. Note the visible color change for both oral sampling swabs (1), wherein a visible color change (e.g., pink color) indicates the presence of bacteria in the obtained biological samples.

### Results

5 test subjects are recruited to evaluate the cleaning performance of the oral care product Crest® Pro-Health toothpaste-Complete 7. Subjects are asked to evaluate their oral cavity cleanliness by using the oral sampling swabs “Before Brush” and “After Brush”. The results are discussed below.

For the “Before Brush” biological samples, the oral sampling swabs exhibited a very intense pink color on the swab head which indicates high amounts of bacteria present in the biological samples. For the “After Brush” biological samples, the oral sampling swabs exhibited a blue (i.e., no color change) or very light pink on the swab head which indicates very low or low amounts of bacteria present in the biological samples. When the “Before Brush” and “After Brush” oral sampling swabs are compared, a substantially notable visual color change (i.e., blue to intense pink) can be easily visualized by the subjects.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical limitations were expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral sampling swab for assessing treatment effectiveness of an oral care product in decreasing the amount of bacteria in an oral cavity, the oral sampling swab comprising:
(a) a swab comprising a handle at one end for handling the swab, a swab head, and a hollow swab stem connecting the handle to the swab head to provide fluid passage of a dye stored in a receptacle inside the handle to the swab head, wherein the swab head is configured for obtaining a biological sample from an oral cavity, wherein the dye is configured to exhibit a visible color change upon reacting with bacteria contained in the obtained biological sample; and
(b) a reaction tube comprising an upper open end and an opposing lower closed end, wherein the swab is removably inserted in the reaction tube such that at least the handle of the swab protrudes from the upper open end when the swab head abuts the lower closed end of the reaction tube.

2. The oral sampling swab of claim 1 wherein the receptacle further comprises a fragible spacer configured to contain the dye in the receptacle and release the dye from storage upon sufficient pressure applied to break the fragible spacer, such that the dye passes from the receptacle through the hollow swab stem into the reaction tube.

3. The oral sampling swab of claim 1 wherein the dye is selected from the group consisting of 7-Hydroxy-3H-phenoxazin-3-one 10-oxide, (3-(4,5-dimethylthiazol-2-yl)-5-(3-carboxymethoxyphenyl)-2-(4-sulfophenyl)-2H-tetrazolium), (2-(4-Iodophenyl)-3-(4-nitrophenyl)-5-(2,4-disulfophenyl)-2H-tetrazolium), (2-(2-methoxy-4-nitrophenyl)-3-(4-nitrophenyl)-5-(2,4-disulfophenyl)-2H-tetrazolium), 3-[4,5-dimethylthiazol-2-yl]-2,5-diphenyltetrazolium bromide, 2,3,5-triphenyltetrazolium chloride, 2-[-iodophenyl]-3-[-dinitrophenyl]-5-phenyltetrazoliumchloride, 2,3-bis[2-methoxy-4-nitro-5-sulfophenyl]-2H-tetrazolium-5-carboxanilide, and combinations thereof.

4. The oral sampling swab of claim 4 wherein the dye comprises 7-Hydroxy-3H-phenoxazin-3-one 10-oxide.

5. The oral sampling swab of claim 3, wherein the dye is further combined with an additional reagent selected from the group consisting of phenazine methyl sulfate, phenazine ethyl sulfate, and combinations thereof.

6. The oral sampling swab of claim 1, wherein the total volume of the dye is from 275 μL to 500 μL.

7. The oral sampling swab of claim 6 wherein the total volume of the dye is from 300 μL to 350 μL.

8. The oral sampling swab of claim 1 wherein the concentration of the dye is from 20 μg/mL to 100 μg/mL.

9. The oral sampling swab of claim 8 wherein the concentration of the dye preferably from 40 μg/mL to 60 μg/mL.

10. The oral sampling swab of claim 1 wherein the overall length of the hollow swab stem is from 7 cm to 11 cm.

11. The oral sampling swab of claim 1 wherein the diameter of the swab head is from 0.35 cm to 0.6 cm.

12. The oral sampling swab of claim 11 wherein the diameter of the swab head is from 0.37 cm to 0.45 cm.

13. The oral sampling swab of claim 1 wherein at least a portion of the reaction tube is translucent or transparent.

14. The oral sampling swab of claim 13 wherein the lower closed end of the reaction tube is translucent or transparent.

15. The oral sampling swab of claim 1 further comprising a snap-lock mechanism for securing the swab to the reaction tube.

16. The oral sampling swab of claim 15 wherein the swab is irreversibly secured to the reaction tube.

17. The oral sampling swab of claim 15 wherein the snap-locking mechanism is formed of a mating locking ring in the upper open end of the reaction tube and a lip integrally formed to a lower portion of the swab handle, such that the insertion of the lip through the locking ring secures the swab to the reaction tube.

18. A method of providing to a consumer a visual demonstration of the treatment effectiveness of an oral care product in decreasing the amount of bacteria in an oral cavity, the method comprising the steps of:
(a) providing an oral sampling swab according to claim 1;
(b) removing the swab from the reaction tube;
(c) obtaining a biological sample of an oral cavity with the removed swab;
(d) returning the swab to the reaction tube with the obtained biological sample;
(e) releasing the dye from the stored receptacle of the returned swab to the reaction tube so as to immerse the swab head in the dye; and
(f) assessing whether the dye exhibits a visible color change or degree of visible color formation as indicative of bacteria amount and a positive assessment of the oral care product's treatment effectiveness.

19. The method of claim 19, wherein the step (c) obtains a biological sample from at least a posterior region of the oral cavity.

20. The method of claim 19, wherein the step (f) assesses the dye visible color change or degree of visible color formation over a period of from 1 minute to 5 minutes.
